(19) United States
(12) Patent Application Publication
(10) Pub. No.: US 2021/0313074 A1
(43) Pub. Date: Oct. 7, 2021

(54) AUTOMATIC CONTACT TRACING

(71) Applicant: PricewaterhouseCoopers LLP, New York, NY (US)

(72) Inventors: Robert MESIROW, Falls Church, VA (US); Patrick PARODI, Bethesda, MD (US); Marc MAZZIE, Washington, DC (US)

(73) Assignee: PricewaterhouseCoopers LLP, New York, NY (US)

(21) Appl. No.: 17/220,375

(22) Filed: Apr. 1, 2021

Related U.S. Application Data
(60) Provisional application No. 63/022,217, filed on May 8, 2020.
(60) Provisional application No. 63/003,714, filed on Apr. 1, 2020.

(51) Int. Cl. G16H 50/80; G16H 50/30; G16H 10/40; G06Q 30/02; H04W 4/02; H04W 4/029

(52) U.S. Cl. CPC G16H 50/80; G16H 50/30; G16H 10/40; H04L 63/083; H04W 4/023; H04W 4/029; G06Q 30/0201

(57) ABSTRACT

Techniques for performing automatic contact tracing using a fleet of mobile electronic devices are provided. By collecting and storing signal data detected by the fleet of devices, the system generates a historical record of timestamped signal data from which interpersonal interactions may be inferred. The system may retrieve information from the stored historical record to determine what users were in contact with a target user during a target time period. The determination may be based on which other devices were proximate to the target device, at what time those other devices were proximate, how close those other devices were, and/or the amount of time for which those devices were proximate. The system may applying one or more algorithms (e.g., a “proximity score” calculation) to the stored record in order quantify and/or characterize a risk for users of devices that were proximate to the target device.

### CROSS-REFERENCE TO RELATED APPLICATIONS

This application claims the benefit of U.S. Provisional Application No. 63/003,714, filed Apr. 1, 2020, and of U.S. Provisional Application No. 63/022,217, filed May 8, 2020, the entire contents of each of which is incorporated herein by reference.

### FIELD

This related generally to systems and methods for automatic contact tracing, and more particularly to automatic contact tracing for tracking potential vectors of disease outbreak.

### BACKGROUND

In order to perform contact tracing to detect and trace potential vectors for disease outbreaks, there is a need to determine when infected and/or potentially-infected persons have come into contact or close proximity with one another. Known methods for contact tracing are labor-intensive and require time-consuming and potentially inaccurate post-hoc manual determination of an infected person's whereabouts and potential interpersonal interactions. Furthermore, known methods of contact tracing provide inadequate privacy protections for participants. Thus, there is a need for improved methods of automatic and accurate contact tracing.

### SUMMARY

Disclosed herein are systems for performing automatic contact tracing using a fleet of mobile electronic devices carried by persons. By collecting and storing signal data detected by the fleet of mobile electronic devices, the system may generate and store a historical record of timestamped device signal data from which interpersonal interactions may be inferred. In the event that a user of the systems then self-reports as having tested positive for a disease (or as having had potential disease exposure), the system may retrieve information from the stored historical record to determine what other users were in contact with that user during the time period during which the self-reporting user may have been contagious, wherein the determination may be based on which other users' mobile devices were proximate to the self-reporting user's mobile device, at what time those devices were proximate to one another, how close those devices were to one another, and/or the amount of time for which those devices were proximate to one another. Based on applying one or more algorithms (e.g., a “proximity score” calculation) to the stored record regarding which users have been in contact with an infected or potentially infected user, the system may quantify and/or characterize a risk for other users, such as an exposure risk, a contamination risk, an infection risk, and/or a disease risk. In some embodiments, a quantification and/or characterization of a risk may include one or both of a numerical score and a categorization into a predefined risk category (e.g., “high risk,” “low risk;” or “high proximity score,” “low proximity score”). The disclosure herein may refer to quantification/characterization of a risk, quantification/characterization of an extent of contact/proximity, and/or calculation of a “proximity score,” and a person of ordinary skill in the art will understand, in light of the disclosure herein, that features of these determinations may be interrelated and may share characteristics with one another. The system may optionally generate one or more notifications based on the calculated risk and/or calculated score. In some embodiments, quantifying and/or characterizing an exposure risk, a disease risk, and/or an extent of contact/proximity may include generating a “proximity score” based on time in proximity, number of times in proximity, and/or closeness of physical distance between devices and/or users.

In some embodiments, a system for performing automatic contact tracing for monitoring disease outbreaks is prided, the system comprising: a plurality of mobile electronic devices, each of the plurality of mobile electronic devices configured to detect signal data indicating when it is proximate to another one of the plurality of mobile electronic devices; one or more processors configured to: receive, from each of the plurality of mobile electronic devices, detected signal data; store a record of the signal data; receive an indication to trace the contacts of a first user associated with a first mobile electronic device of the plurality of electronic devices; in response to receiving the indication to trace the contacts of the first user, retrieve at least a portion of the signal data from the record, the retrieved signal data indicating which of the plurality of mobile electronic devices have been proximate to the first mobile electronic device; and generate, based on the retrieved signal data, a quantification of risk for one or more other users associated with one or more of the plurality of mobile electronic devices that have been proximate to the first mobile electronic device.

In some embodiments, the indication to trace the contacts of the first user comprises an indication that the first user has tested positive for a disease.

In some embodiments, the indication to trace the contacts of the first user comprises an indication that the first user has potentially been exposed to a disease.

In some embodiments, each of the plurality of mobile electronic devices is configured to broadcast electromagnetic signals comprising identifying information able to be detected by one or more of the other mobile electronic devices.

In some embodiments: the signal data received from each of the plurality of mobile electronic devices comprises time data indicating when pairs of the mobile electronic devices were proximate to one another; and storing the record of the signal data comprises storing the time data.

In some embodiments: the indication to trace the contacts of the first user comprises an indication of a time window for which contact tracing should be performed; and the retrieved signal data is retrieved based on the time data stored in the record indicating that one or more of the plurality of mobile electronic devices was proximate to the first mobile electronic device during the time window indicated.

In some embodiments, the system comprises: a first database in which the record of signal data is stored in association with user identifiers; and a second database in which user personal information is stored in association with the user identifiers.

In some embodiments, the one or more processors are configured to, in response to receiving the indication to trace the contacts of the first user: retrieve a first user identifier associated with the first user from the second database; and retrieve the signal data comprises retrieving the signal data from the first database using the first user identifier.

In some embodiments, the user identifiers comprise user identifiers configured for use in advertisement systems.

In some embodiments, the one or more processors are configured to generate and transmit one or more notifications regarding the quantification of risk for one or more users.

In some embodiments, the quantification of risk is based on an amount of time for which the mobile electronic devices of the first user and the one or more other users were proximate to one another.

In some embodiments, the quantification of risk is based on a number of times that the mobile electronic devices of the first user and the one or more other users were proximate to one another.

In some embodiments, the quantification of risk is based on a calculated distance between the mobile electronic devices of the first user and the one or more other users that were proximate to one another.

In some embodiments: each of the plurality of mobile electronic devices is configured to detect signal data when predefined location criteria are satisfied; and each of the plurality of mobile electronic devices is configured to not detect signal data when the predefined location criteria are not satisfied.

In some embodiments, the one or more processors are configured to: receive, from each of the plurality of mobile electronic devices, location data associated with the detected signal data; store the record of the signal data in accordance with a determination that predefined location criteria are satisfied; and not store the record of the signal data in accordance with a determination that the predefined location criteria are not satisfied.

In some embodiments, the one or more processors are configured to automatically delete the received location data after determining whether the location criteria are satisfied and without regard for whether or not the location criteria are satisfied.

In some embodiments, the detected signal data comprises information about a detected signal broadcast from another of the plurality of mobile electronic devices.

In some embodiments, the detected signal data comprises information about a detected signal broadcast from a device, separate from the plurality of mobile electronic devices, including one or more of: a Wi-Fi access point, a Bluetooth device, a network-enabled appliance, a network-enabled infrastructure device, and an IoT devices.

In some embodiments, the record of signal data comprises, for each of the plurality of electronic devices, a signal profile generated based on a plurality of electromagnetic signals detected by a set of one or more detection antennas of the mobile electronic device.

In some embodiments, the plurality of electromagnetic signals comprise signals emitted one or more of: a Wi-Fi access point, a Bluetooth device, a network-enabled appliance, a network-enabled infrastructure device, an IoT device, and another of the plurality of mobile electronic devices.

In some embodiments, wherein the detected signal data comprises: a first subset of the signal data collected in accordance with a first type of signal scan performed according to a first timing schema; and a second subset of the signal data collected in accordance with a second type of signal scan performed according to a second timing schema.

In some embodiments, the quantification of risk comprises one or both of: a numeric score, and a classification into a predefined risk category.

In some embodiments, the quantification of risk comprises one or more of: an exposure risk, a contamination risk, an infection risk, and a disease risk.

In some embodiments, generating the quantification of risk comprises: generating a vector comprising a plurality of vector components, wherein each of the plurality of vector components corresponds is computed based on comparing the signal data for to a respective predefined component threshold; calculating a weighted sum of the vector components; comparing the weighted sum of the vector components to a predefined threshold to determine a risk category.

In some embodiments, a method for performing automatic contact tracing for monitoring disease outbreaks is provided, the method performed at a system comprising one or more processors and a plurality of mobile electronic devices, each of the plurality of mobile electronic devices configured to detect signal data indicating when it is proximate to another one of the plurality of mobile electronic devices, the method comprising: receiving, from each of the plurality of mobile electronic devices, detected signal data; storing a record of the signal data; receiving an indication to trace the contacts of a first user associated with a first mobile electronic device of the plurality of electronic devices; in response to receiving the indication to trace the contacts of the first user, retrieving at least a portion of the signal data from the record, the retrieved signal data indicating which of the plurality of mobile electronic devices have been proximate to the first mobile electronic device; and generating, based on the retrieved signal data, a quantification of risk for one or more other users associated with one or more of the plurality of mobile electronic devices that have been proximate to the first mobile electronic device.

In some embodiments, a non-transitory computer-readable storage medium comprising instructions for performing automatic contact tracing for monitoring disease outbreaks at a system at comprising one or more processors and a plurality of mobile electronic devices is provided, each of the plurality of mobile electronic devices configured to detect signal data indicating when it is proximate to another one of the plurality of mobile electronic devices, the instructions configured to cause the system to: receive, from each of the plurality of mobile electronic devices, detected signal data; store a record of the signal data; receive an indication to trace the contacts of a first user associated with a first mobile electronic device of the plurality of electronic devices; in response to receiving the indication to trace the contacts of the first user, retrieve at least a portion of the signal data from the record, the retrieved signal data indicating which of the plurality of mobile electronic devices have been proximate to the first mobile electronic device; and generate, based on the retrieved signal data, a quantification of risk for one or more other users associated with one or more of the plurality of mobile electronic devices that have been proximate to the first mobile electronic device.

In some embodiments, any one or more of the features of any one or more of the embodiments set forth above may be combined with one another, and/or with other features or aspects of any method, system, technique, or device disclosed herein. The subject matter of this application may involve, in some cases, interrelated products, alternative solutions to a particular problem, and/or a plurality of different uses of a single system or article.

### BRIEF DESCRIPTION OF THE FIGURES

The patent or application file contains at least one drawing executed in color. Copies of this patent or patent application publication with color drawings will be provided by the office upon request and payment of the necessary fee.

FIG. 1 shows a schematic representation of a system for automatic contact tracing, in accordance with some embodiments.

FIG. 2A shows an example of multiple mobile electronic devices deployed in a system, in accordance with some embodiments.

FIG. 2B shows an example of a heat map of a proximity score calculated based on duration of physical proximity and on closeness (distance) of physical proximity.

FIG. 3 shows a flowchart showing a method for performing automatic contact tracing, in accordance with some embodiments.

FIG. 4 shows a screen of a graphical user interface for authorized users to input, view, and access information about infected in accordance with some embodiments.

FIG. 5 shows a screen of a graphical user interface for authorized users to input, view, and access information about potentially exposed employees, in accordance with some embodiments.

FIG. 6 shows a computer, in accordance with some embodiments.

These and other features of the present embodiments may be understood better by reading the following detailed description, taken together with the figures herein described. In the drawings, identical or nearly identical components illustrated in multiple figures may be represented by a like reference numeral. For purposes of clarity, not every component may be labeled in every drawing. Furthermore, as will be appreciated in light of this disclosure, the accompanying drawings are not intended to be drawn to scale or to limit the described embodiments to the specific configurations shown.

### DETAILED DESCRIPTION

As described above, in some embodiments, an automatic contact tracing system collects and stores signal data detected by a plurality of mobile electronic devices, and the system may generate and store a historical record of timestamped device signal data from which interpersonal interactions may be inferred. In the event that a user of the systems then self-reports as having tested positive for a disease (or as having had potential disease exposure), the system (e.g., in response to a user request) may retrieve information from the stored historical record to determine what other users were in contact with that user during the time period during which the self-reporting user may have been contagious, wherein the determination may be based on which other users' mobile devices were proximate to the self-reporting user's mobile device, at what time those devices were proximate to one another, how close those devices were to one another, and/or the amount of time for which those devices were proximate to one another. Based on applying one or more algorithms such as a proximity score calculation to the stored record regarding which users have been in contact with or proximate to an infected or potentially infected user, the system may quantify and/or characterize an exposure risk, disease risk, and/or proximity score for other users. The system may optionally generate one or more notifications based on the calculated exposure risk, disease risk, and/or proximity score. In some embodiments, quantifying and/or characterizing an exposure risk, a disease risk, and/or an extent of contact/proximity may include generating a proximity score based on time in proximity, number of times in proximity, and/or closeness of physical distance between devices and/or users. In some embodiments, the mobile electronic devices (which may be, for example, personal cell phones, wearable devices, or the like) leveraged in the system may be configured to automatically detect signal data that may be used to determine physical proximity (e.g., closeness of distance) to other mobile electronic devices. The mobile electronic devices may in some embodiments be devices issued to employees by an employer, and in some embodiments may be configured by an over-the-air software update to perform one or more of the contact tracing functionalities described herein. In some embodiments, the mobile electronic devices may include cell phone devices (e.g., company-issued cell phones or personal cell phones); in some embodiments, the mobile electronic devices may include a dedicated/standalone device configured to broadcast, collect, and upload signals for use in proximity score determination, risk determination, and/or contact tracing as discussed herein.

In some embodiments, the mobile electronic devices may be configured to detect (e.g., to “listen for”) electromagnetic signals emitted by other users' mobile electronic devices. Said electromagnetic signals, such as WiFi signals and/or Bluetooth signals, may only be able to be detected when the emitting device and the listening device are within a certain physical proximity (e.g., a certain physical distance) of one another, and their detection may therefore be taken as an indication that the devices are within a certain estimated physical proximity (e.g., a certain estimated physical distance) of one another at the time of the detection. In some embodiments, detection of a signal from another mobile electronic device may be noted; in some embodiments, a strength of a signal detected from another mobile electronic device may be noted, such that the signal strength may thereafter be used to inform a calculation of estimated physical proximity (e.g., estimated distance) between the two devices.

In some embodiments, mobile electronic devices in the system may also be configured to actively broadcast one or more signals (for example indicating a MAC address, Beacon ID, AdID, user identifier, and/or other identifier of the device) for other participating mobile electronic devices to detect. In some embodiments, mobile electronic devices configured for participation in a contact tracing system such as those disclosed herein may operate as a Bluetooth low-energy (BLE) beacon in order to broadcast information (continuously and/or periodically) about the device's MAC address, AdID, Beacon ID, an identifier identifying the contact tracing system and/or platform, and/or an identifier for the specific mobile electronic device itself.

Alternatively or additionally, rather than taking the detection itself as an indicator of estimated physical proximity (e.g., physical distance), signals that are detected by a listening device may be used to construct a “signal map” for the listening device, and the signal map may be used to calculate estimation of the listening device's physical proximity (e.g., physical distance) to one or more other electronic devices based on the strength and identity of the signals detected. The signal map may comprise information about the identity, strength, configuration, and timing of signals detected by a mobile electronic device.

Alternatively or additionally still, in some embodiments, the mobile electronic devices may be configured to detect electromagnetic signals emitted by electronic devices that are not mobile electronic devices of another user. For example, a mobile electronic device may detect signals, such as WiFi signals and/or Bluetooth signals, emitted by a WiFi hot-spot or IOT device. In some embodiments, information about signals detected from these devices may be stored (e.g., in a user's signal map and/or in a database of signals data detected by devices in the system) and used to determine a user's physical proximity (e.g., physical distance) to another user. For example, if two users' mobile devices are each connected to the same WiFi hot-spot at the same time, then the system may determine that the users are in close physical proximity (e.g., close physical distance) to one another (e.g., within a distance of at most twice the estimated signal range of the WiFi hot-spot), even if neither of those users' mobile devices detect any signals emitted by one another directly.

In some embodiments, mobile electronic devices participating in the contact tracing system may be configured to perform two kinds of signal scans for the purpose of collecting signals to be used in building a database of signal data. First, each mobile electronic device may perform periodic scans for signals, such as ambient WiFi signals and/or Bluetooth signals, emitted by other devices such as WiFi hot spots, IOT devices, or the like. For example, a device may periodically (e.g., every 10 minutes or at any other predetermined interval or in accordance with dynamic triggering) scan ambient WiFi and Bluetooth signals and send signal scan reports along with the device's AdID, user identifier (e.g., UUID), unique BLE identifier, and/or location information to a server associated with data storage and/or contact analysis.

Second, each mobile electronic device may perform continuous (or, in some embodiments, intermittent or periodic) ranging (scans) of BLE signals transmitted by other mobile electronic devices configured for participation in the contact tracing system. (Each mobile device may itself transmits BLE (Bluetooth Low Energy) signals such that the devices in the system may use phone-to-phone beaconing to detect other nearby devices in the system.) The BLE signals transmitted by those other mobile electronic devices may comprise identifiers identifying the contract tracing system and/or platform and/or identifying the broadcasting mobile electronic device itself. BLE signals detected by other mobile electronic devices may, in some embodiments, provide more direct information regarding the device generating the BLE signal usable in addition to the information about other (outside the system) mobile electronic devices detected by the first scan described above. In some embodiments, signals detected by one kind of scan may be weighted more heavily than signals detected by the other kind of scan in proximity score calculations and/or risk calculations; for example, BLE signals data from mobile electronic devices within the system may be weighted more heavily than signal data from devices not within the system. BLE signal scan reports (along with the uploading user's AdID and/or UUID and location information) may be sent to the server associated with data storage and/or contact analysis. Upload of BLE signal scan reports and associated information may be performed periodically, such as every 1-2 minutes.

As discussed below, location information uploaded in relation to one or both scanning operations may, in some embodiments, be used only for geofencing purposes and not for physical proximity (e.g., physical distance) determination purposes or for proximity score determination purposes.

Information about signal data (whether in the form of a signal map or in any other format) may be transmitted from the mobile electronic devices to a database for storage of the signal data. In some embodiments, signal data may be uploaded for storage in a database by WiFi connection or by other internet connection. In some embodiments, signal data may be stored along with associated metadata (e.g., time of detection, time of storage, device type used to collect the data, etc.). In some embodiments signal data may be stored in association with a user identifier such as an identifier used to identify users, devices, or other assets for the purpose of advertisement services (e.g., an AdID and/or a UUID). In some embodiments, signal data may be stored separately from personal information about users (e.g., user name, user address, user demographic information, etc.), such as by maintaining personal information in a separate database. In some embodiments, signal data may be maintained by a third-party service (e.g., a separate organizational entity, a separate cloud provider, and/or a separate network infrastructure) that does not have access to user personal information, in order to increase user privacy. As used herein, upload and storage of signal data may refer to upload and storage of data regarding detected signals (e.g., signal identity, signal content, signal type, signal strength, time of detection). This data regarding detected signals may thereafter be used to calculate a proximity score indicating information about whether one or more devices (or associated persons) are estimated to have been in contact with or proximate to one another.

After a record of the detected signal data has been stored, the system may then perform contact tracing upon request for one or more users. In some embodiments, performing contact tracing for one or more users may be triggered by the system receiving a request to trace contacts of a specific user. A request to trace contacts of a specific user may be triggered by the system receiving an indication that the user has tested positive for a disease, that the user has been exposed to someone who has tested positive for a disease, or that the user otherwise has a particularized risk of disease. The system may be configured to accept self-reporting for positive disease testing or potential exposure from users and/or to allow system administrators to execute an input indicating a positive test or potential exposure for a user.

In some embodiments, the system may provide a web-portal or other interface (e.g., a dashboard) for enterprise-level users (e.g. human-resources administrators, in the example of an enterprise-deployed system) to input, view, and access information about infected and/or potentially exposed employees and any results of contact tracing performed for said employees. Enterprise-level dashboard users may be able to configure one or more enterprise-wide and/or user-specific data collection preferences, for example in order to comply with different data privacy regulations in different regions.

In some embodiments, the system may be configured such that contact tracing is performed upon receipt of a request to trace contacts of a specified device/person (in some embodiments, during a specified time), wherein the request may be executed by a user of the web-hosted dashboard provided by the system. In some embodiments, the system may be configured such that contact tracing is not initiated automatically, but is only performed when a dashboard user inputs an instruction to trace contacts for a specified person. In this way, when a person/employee self-reports as exposed or potentially exposed (e.g., through human-resources channels maintained outside the contact tracing system), the dashboard user may then input an instruction to the dashboard for the contact tracing system to trace contacts for the exposed or potentially exposed user, wherein the input includes personal identifying information for the user. A request to perform contact tracing may include identifying information for a user for whom to trace contacts (e.g., name or email address) and a date/time range over which contacts for that user should be traced.

Upon receiving an input from a user (e.g., from a dashboard user or from any other enterprise-level administrator) indicating that the system should trace the contacts of a user, the system may retrieve some of all of the signal data for the user from the database storing the signal data. In some embodiments, retrieving the signal data for a user may require the system to first obtain the user identifier (e.g., AdID and/or UUID) used by the database storing the signal data, such that the system may identify the relevant signal data to extract from the database. In some embodiments, the system may look up the identifier associated with the user in a separate database, such as an enterprise database maintained separately from the database storing the signal data.

In order to trace the contacts of a user, the system may retrieve signal data associated with the target user over a time period associated with the target user's infection or potential exposure. For example, a request to trace user contacts may specify a period of time over which contacts should be traced, and signal data associated with that time window may then be retrieved. In some embodiments, the system may determine a window over which to retrieve signal data based on a disease diagnosis or potential disease exposure for the user; for example, the time period may be determined based on an incubation period or potential incubation period of a disease for which a user has tested positive.

Once signal data for the target user has been retrieved, the system may use the signal data to determine which other users (e.g., which other user devices) have been proximate to the target user during the target time period. For each such proximate user, the system may calculate a quantification and/or characterization of risk (e.g., a quantification of exposure risk, contamination risk, infection risk, and/or disease risk), such as a numerical risk score (e.g., a “proximity score”), a risk classification (e.g., high risk, medium risk, low risk), and/or a proximity classification (e.g., high proximity, medium proximity, low proximity). In some embodiments, a quantification and/or characterization of risk (e.g., exposure risk or disease risk) may include and/or be provided as a quantification and/or characterization of time in proximity and/or closeness of physical proximity, such as a proximity score. The quantification and/or characterization of risk and/or proximity score may be calculated in accordance with one or more predefined algorithms and/or using one or more machine learning algorithms. In some embodiments, the calculation may be based on a number of times a user was proximate to an exposed user, a time at which a user was proximate to an exposed user, a length of time over which a user was proximate to an exposed user, and/or a closeness of physical proximity (e.g., closeness of physical distance, for example calculated and/or inferred based on signal strength) between a user and an exposed user and/or between devices with which the user and the exposed user are associated. In some embodiments, a quantified and/or characterized risk may be determined with respect to a specific disease and/or pathogen, and the system may be configured to calculate different risks (e.g., exposure risks, contamination risks, infection risks, disease risks) for a single user for different diseases and/or pathogens.

In some embodiments, the system may be configured to apply one or more algorithms to calculate a risk level and/or proximity score to determine which users should be classified as having a high proximity score, medium proximity score, or low proximity score with respect to the target user. In some embodiments, the calculated proximity score may be a function of one or both of the duration and physical proximity (e.g., physical distance) of detected-signal overlap and/or cross-device signal detection for the two users. For example, duration of contact may be estimated based on observed overlapping time between two mobile electronic devices and may be incorporated with frequency of overlap. Physical proximity (e.g., physical distance) may be estimated based on signal strength (e.g., RSSI) of BLE signals transmitted by nearby mobile electronic devices. If a second user's mobile electronic device observes a similar set of ambient WiFi and/or Bluetooth signals as an infected user's mobile electronic device for an overlapped time, or the second user's mobile electronic device detects the infected user's BLE signal, the system may determine that the second user has been in contact with the infected user. In some embodiments, a second user may be determined to be at a higher risk (e.g., higher proximity score) if the second user's mobile electronic device detected similar and/or overlapping signals with an infected user with a higher signal strength and/or for a longer period of time.

In one example, proximity characterizations and/or risk characterizations may classify one or more users/devices into “high proximity score,” “medium proximity score,” and “low proximity score” categories as follows. A high proximity score may be assigned when a second user's mobile electronic device scanned similar ambient WiFi/Bluetooth signals as those signals scanned by a target user's mobile electronic device, and/or the second user's mobile electronic device received a strong signal (e.g., above a predetermined or dynamically determined signal strength threshold) via BLE transmission from a target user's mobile electronic device for more than a predetermined or dynamically determined threshold amount of time (e.g., 30 minutes), with the threshold amount of time calculated either continuously or intermittently (e.g., cumulatively allowing for interruptions), during a date/time range specified via the system dashboard. A medium proximity score may be assigned when a second user's mobile electronic device detects signals that overlap with signals detected by a target user (e.g., overlapping in identity, strength profile, and/or time) in a significant manner (e.g., exceeding a signal strength threshold and/or a time threshold) in a first instance but in an insignificant manner (e.g., not exceeding one or both of a signal strength threshold and/or a time threshold) in a second instance. A medium proximity score may also be assigned when a signal strength of a BLE signal detected from a target user's mobile electronic device is strong in one instance (e.g., exceeding a strength threshold) but is weak in another instance. A low proximity score may be assigned when ambient WiFi/Bluetooth/BLE signals detected by a second user's mobile electronic device have any non-zero signal overlap with the signal scans detected by (or BLE signals broadcast from) the target user's mobile electronic device within a predefined or dynamically determined time window (e.g., 4 hours). (Thus, in some instances, users who were never in the same place at the same time may nonetheless be assigned a low proximity score if they were in the same place within a threshold time window of one another.) In some embodiments, users not meeting the criteria for high, medium, or low proximity score may not be assigned any proximity score.

In some embodiments, the system may generate one or more notifications regarding one or more of the users who have been proximate to an exposed or infected user. For example, the system may generate a report with respect to all users who have been in contact with an exposed user, and/or may generate an alert/waning to users whose risk (e.g., disease risk, exposure risk, and/or proximity score) meets predefined risk criteria (e.g., for users whose risk is classified as “medium risk” or “high risk” and/or whose proximity scores are classified as “high” or “medium”). In some embodiments, the one or more notifications may be stored and/or transmitted to system administrators (e.g., enterprise dashboard users), to users to whom a notification pertains, and/or to public health officials. In some embodiments, notifications may be automatically pushed to the mobile electronic device of a user who meets alert/warning criteria.

In some embodiments, the system may be configured such that output data regarding a contact tracing query may be displayed to and/or outputted for a user of the system, such as an enterprise-level dashboard user, and may thereafter be destroyed. For example, in some embodiments, alerts may be sent to potentially exposed users through preexisting enterprise channels outside the contract tracing system, rather than being electronically and/or automatically being transmitted to potentially exposed users through the contact tracing system itself. In some embodiments, results of a contact tracing query, including a list of potentially exposed users and/or quantifications/characterizations of risk (e.g., proximity scores) for one or more users, may not be persistently stored in the system. For example, a list of potentially exposed users may be automatically deleted by the system when a dashboard session is terminated.

In some embodiments, in addition to or alternatively to reporting specific identifiable persons who have potentially been exposed, the system may generate anonymized data regarding exposure extent to be transmitted to public health officials and/or to one or more third-party organizations. In some embodiments, data may be anonymized for display regarding enterprise-wide and/or population-wide exposure extent by hashing personally-identifiable information. In some embodiments, the system may be configured to display one or more statistics and/or metrics regarding enterprise-wide and/or population-wide exposure extent, determined in accordance with applying the contact tracing algorithm. For example, the system may display (and/or transmit) a map view of directly reported cases and/or potential exposures of other users to those users who have directly reported exposure. In another example, the system may display summary statistics such as directly reported cases, total potential exposures, locations affected, high risk employees, total potential exposure, and/or offices affected.

In some embodiments, the system may be configured to perform second-order contact tracing (or third-order contact tracing) by which contacts may be automatically traced (and/or traced upon request) not only for a target user who has been designated as exposed or potentially exposed, but also for those users who have come into contact with the target user. Higher-order contact tracing may be performed for highly contagious diseases and/or in order to comply with highly conservative contact tracing protocols.

In some embodiments, to improve user privacy and/or to comply with data privacy regulations, one or more functionalities of the system described herein may be geo-fenced (e.g., configured to function only in certain geographic locations, or configured to not function in certain geographic locations). For example, in some embodiments in which an automatic contact-tracing system is implemented in an enterprise environment using company-issued mobile electronic devices to trace potential disease contacts for employees, the mobile electronic devices may be configured to broadcast a signal and/or to gather signal data only when the devices are located on company grounds. In some embodiments, the system may be configured such that mobile electronic devices gather and upload signal data regardless of location, but that the signal data is uploaded along with location data indicating a location of the uploading mobile electronic device, such that the signal data may be stored only when the location data indicates that the uploading mobile electronic device is in an approved area such as on company grounds. (In some embodiments in which location data is uploaded for the purposes of verifying that geo-fencing criteria is met, the uploaded location data may be deleted and may not be stored persistently by the system.) In some embodiments, detected proximity to company-associated electronic devices (e.g., based on signal overlap with and/or signal detection to or from WiFi devices and/or IOT devices) may be used to determine that a mobile electronic device is on company grounds; in some embodiments, GPS data may be used to determine that a mobile electronic device is on company grounds. In another example, functionality may be restricted or disabled in certain sensitive areas (e.g., government facilities, military facilities) and/or in different legal jurisdictions (e.g., different states or countries).

In some embodiments, to improve user privacy and/or to comply with data privacy regulations, one or more functionalities of the system described herein may be configured to only function during certain times (or to not function during certain times). For example, in some embodiments in which an automatic contact-tracing system is implemented in an enterprise environment using company-issued mobile electronic devices to trace potential disease contacts for employees, the mobile electronic devices may be configured to broadcast a signal and/or to gather signal data only during business hours and/or only during times when a specific employee is scheduled for work and/or clocked in for work. In some embodiments, the system may be configured such that mobile electronic devices gather and upload signal data regardless of time, but that the signal data is uploaded along with time data indicating a time of the data collection, such that the signal data may be stored only when the time data indicates that an employee associated with the uploading mobile electronic device is scheduled for work and/or clocked in for work at that time.

In some embodiments, in order to protect participant privacy and/or to comply with data privacy regulations, signal data may be deleted after the expiration of a predefined time period and/or after one or more notifications of potential risk have been displayed, exported, and/or distributed to affected users. In some embodiments, in order to protect participant privacy and/or to comply with data privacy regulations, calculated contact-tracing results data (e.g., determination of characterizations and/or quantifications of proximity/risk/exposure/disease) may be deleted after the expiration of a predefined time period and/or after one or more notifications of potential risk have been displayed, exported, and/or distributed to affected users. For example, signal data and/or contact tracing results data may be deleted after a period of time has passed, for example a period of time that is longer than an incubation period of a disease. In another example, contact tracing results data may be automatically deleted immediately after being output and/or transmitted, or may be deleted when an enterprise-level dashboard user terminates a session. In another example, contact tracing information for an exposed or potentially-exposed user may be deleted after contact tracing has been performed and notifications have been displayed, output, and/or sent to all potentially compromised users. In some embodiments, stored data (e.g., signal data) may be queried in real-time and search results may never be stored persistently in the system. In some embodiments, authorized enterprise-level dashboard users may download contact tracing data, but the data contact tracing data (e.g., search result data) may not be persistently stored in the system.

In some embodiments, the system may be configured to treat one or more fixed electronic devices aside from a mobile electronic device as a hub for the purposes of contact tracing. For example, in addition to enabling mobile electronic devices to broadcast identity information and to listen for signals emitted by other devices, the system may alternatively or additionally enable one or more fixed electronic devices to broadcast identity information and/or to listen for signals emitted by other devices. Fixed electronic devices may include, for example, desktop computers, WiFi hot spots, routers, AV equipment, IOT devices, or the like; in some embodiments, a fixed electronic device may be identified based on a Wireless AP MAC ID, rather than a mobile device UUID or an AdID. In this manner, fixed electronic devices may collect signal data that may be used for contact tracing, wherein a fixed physical space (e.g., a conference room in which a certain AV device is located) may be treated as an entity for the purpose of contact tracing. In this way, contacts between pairs of human users associated with respective mobile electronic devices may be traced, and contacts between a human user and a physical space or fixed electronic device may also be similarly traced using the methods described herein.

In some embodiments, the system may be deployed as a web-based software-as-a-service system in which contact tracing functionality is provided to one or more enterprise users. For example, the system may include a one or more back-end analysis engines for applying contact tracing algorithms and/or one or more back-end databases for storing uploaded signal data. An enterprise user may be onboarded for use of the system by providing the back-end system with geofencing location information for the enterprise and with information regarding authorized enterprise-level users who should be allowed to access the enterprise-specific dashboard interface for applying the contact tracing functionality. One or more authorized enterprise-level dashboard users may then use the dashboard interface to upload employee data to be stored on an employee information database (which may be maintained separately from the signal data database).

In some embodiments, the system may include a web application that acts as a broker between the employee information database and one or more servers or processors for applying the contact tracing algorithm(s). The web application may be cloud-hosted and may process information inputted as part of a dashboard query by a dashboard user. For example, the web application may map an email address of a target user (inputted by the dashboard user) to the UUID and/or AdID of the target user's mobile electronic device and may pass the UUID and/or AdID (along with a date/time range inputted by the dashboard user as part of a query) to the one or more servers for performing contact tracing. When contact tracing results are returned by the one or more servers, the web application may then use the employee information database map the UUIDs and/or AdIDs of the mobile electronic devices indicated as having been in contact with the target user's device to user information (e.g., names, email addresses, and/or offices) for users of those other devices. The web application may then transmit the contact tracing analysis results to the dashboard for visualization. In some embodiments, the contact analysis results data is not stored in the web application, nor in any database of the system, but is instead automatically discarded as soon as the dashboard closes the window of the analysis results or otherwise terminates their session.

Individual employees/users within an enterprise (e.g., employees carrying a mobile electronic device and participating in signal broadcasting/collection) may be onboarded by installing an application associated with the system onto their mobile electronic device, which may then upload the device's unique UUID and/or AdID or other unique identifier to be stored in the employee information database in correlation with the employees personal identifiable information (e.g., name, email address, etc.).

In some embodiments, the system may provide functionality for tracking adoption by employees or other eligible users, for example by tracking which employees or eligible users have downloaded and enabled an application for participating in the system. In some embodiments, enterprise-level dashboard users may be able to access a list of employees who have or have not installed and enabled an application for participation in the system.

In some embodiments, the system may be configured in accordance with one or more data segregation principles in order to increase user privacy. For example, the system architecture may be designed such that personal data access is segregated and restricted based on controlled, limited business needs. In some embodiments, system-wide administrator-level controls (e.g., back-end controls potentially applicable across a plurality of different enterprise-level deployments) may control user role-based access privileges. In some embodiments, databases may be physically separated in multi-tenant architecture in order to segregate data.

In some embodiments, encryption of data while in transit and/or at rest may be used to increase use privacy and data security.

In some embodiments, the system may be configured in accordance with one or more data minimization principles in order to increase user privacy. For example, individual user data used by the system may be limited to employee name, office location, employee ID, email address, and mobile device UUID (or other identifier). Signal data may, in some embodiment, be limited to signal data based on scanned ambient WiFi/Bluetooth signals and/or BLE signals and proximity scores. (As explained elsewhere herein, individual user data may be stored separately from signal data (for example by being stored in a separate database, by a separate entity/service, and/or in a separate cloud instance) and may be linked with one another only in response to receiving a request for the system to perform contact tracing for a user.) In some embodiments, contact tracing may only be performed when requested by authorized personnel (e.g., dashboard users) and contact tracing data may be destroyed immediately after the tracing session is completed. In some embodiments, results of contact tracing queries may be visible only to enterprise-level dashboard users, and may not be visible to back-end system administrators. In some embodiments, the system may be configured such that uploaded location data is not persistently stored and is not shared with or available to enterprise-level users (e.g., dashboard users).

In some embodiments, the system may be configured in accordance with one or more data deletion principles in order to increase user privacy. For example, signal data may be automatically and/or periodically deleted at regular and/or dynamically determined intervals, such as 15 days, 30 days, or 45 days. The amount of time after which data is automatically deleted may be determined in accordance with incubation period, infection periods, and/or outbreak periods for a particular contagion. In some embodiments, the system may be configured such that back-end administrators have data deletion privileges that may be applied, responsive to user or government requests, to enterprise deployments.

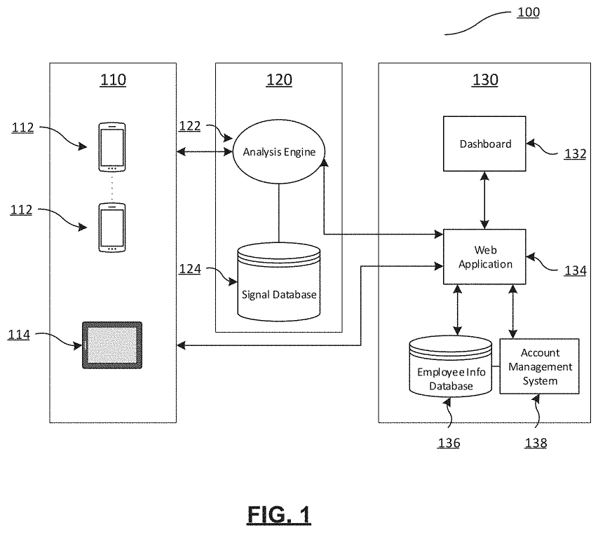

FIG. 1 shows a schematic representation of a system 100 for automatic contact tracing (such as for tracking potential vectors of disease outbreak), in accordance with some embodiments. While systems such as system 100 may be referred to herein as systems for tracking potential vectors of disease outbreak in a group of employees, a person of skill in the art will appreciate in light of the disclosure herein that systems such as those disclosed herein may be readily adapted for use in any context that may require contact tracing in any group of users (e.g., students in school, members of an organization, etc.). In some embodiments, systems 100 may be configured to provide any of the data collection functionality, data analysis functionality, contact tracing functionality, and/or any other functionality described above and/or elsewhere herein.

In the example of FIG. 1, system 100 comprises a plurality of mobile electronic devices 110. In some embodiments, the plurality of mobile electronic devices may be devices 110 issued to employees by an employer, and in some embodiments may be configured by an over-the-air software update to perform one or more of the contact tracing functionalities described herein. In some embodiments, the mobile electronic devices 110 may include cell phone devices 112. In some embodiments, the mobile electronic devices 110 may include a standalone device 114 configured to broadcast, collect, and upload signals for use in proximity score determination and contact tracing as discussed herein.

In some embodiments, each of a plurality of mobile devices are identifiable by system 100, such as by a unique device identifier, a unique user identifier, or other metadata. In this way, the signal data of multiple persons or mobile electronic devices may be tracked and stored. For the purposes of illustration, this disclosure may refer to characteristics of mobile electronic device 112, but a person of ordinary skill in the art will appreciate in light of the disclosure herein that those characteristics may be shared, in some embodiments, by one or more additional devices that may be in a plurality of mobile electronic devices 110 of system 100.

In some embodiments, mobile device 112 may comprise one or more antennas configured to detect electromagnetic signals emitted by devices in the environment or by one or more other devices in the plurality of mobile electronic devices 110. The electromagnetic signals may comprise signals emitted by one or more of: Wi-Fi access points, Bluetooth devices, network-enabled appliances, network-enabled infrastructure devices, IoT devices, and another of the plurality of mobile electronic devices.

As device 112 moves about different locations, it may detect signals from different devices in the one or more mobile electronic devices 110, and may detect those signals at different strengths; the identity and characteristics of those signals detected by device 102 may be used, as described herein, to determine time in proximity and/or distance of physical proximity of device 112 to other mobile electronic devices in the one or more mobile electronic devices 110.

In some embodiments, mobile electronic device 112 may comprise one or more antennas configured to emit electromagnetic signals. The electromagnetic signals emitted by mobile electronic device 112 may comprise one or more of: Bluetooth/BLE signals, Wi-Fi hot spot signals, signals indicating a MAC address, and/or signals indicating a UUID and/or an AdID of the device.

System 100 may further comprise proximity analysis system 120. Proximity analysis system 120 may be any system or server (or plurality of servers) that may communicate with other components of system 100 by electronic network communication. In some embodiments, analysis system 120 may be disposed remotely from one or more of devices 110. In some embodiments, proximity analysis system 120 may include one or more cloud-based systems, or may be provided in whole or in part by one or more cloud-based systems. Proximity analysis system 120 may be configured to execute one or more processes, as explained herein, to receive signal data of one or more mobile devices 110, process the information received, and generate information regarding determined risk level (e.g., a proximity score) of the one or more mobile devices 110 (or for one or more users associated therewith).

Proximity analysis system 120 comprises a analysis engine 122. Analysis engine 122 comprises one or more computer processors that execute instructions to perform any one or more of the techniques disclosed herein, including but not limited to receiving and processing signal data in order to perform contact tracing.

Proximity analysis system 120 also comprises one or more computer storage devices (e.g., databases) configured to provide a signal database 124 configured to store information regarding the received signal data of the one or more mobile devices in 110, such as signal types, signal strengths, signal information content, signal duration, and/or time of collection or other metadata regarding signals detected by one or more of devices 110. In some embodiments, database 124 may also store instructions for performing contact tracing methods (e.g., risk determinations including proximity score calculations) as disclosed herein and/or data (e.g., configurations and/or settings for systems and/or devices, historical logs, etc.) regarding one or more proximity analysis system such as system 120; in some embodiments, said instructions and/or data may be stored separately from databased 124. Signal database 124 may store signal data regarding detected signals (e.g., signal identity, signal content, signal type, signal strength, time of detection, etc.). This data regarding detected signals may be used to calculate a quantification or to generate a characterization, such as calculating a proximity score, indicating information about whether one or more devices (or associated persons) are estimated to have been proximate to one another and/or whether one or more persons are estimated to have been subjected to a risk (e.g., an exposure risk).

In some embodiments, signal data may be maintained by a third-party service that does not have access to user personal information (and/or may otherwise be maintained in a separate physical infrastructure and/or separate cloud infrastructure), in order to increase user privacy. (For example, user personal information may be stored in employee information database 136, which may be physically segregated from database 124 and may be secured via different sets of user permissions.

In some embodiments, the one of more mobile devices 110 may send geolocation information (e.g., device location data) to the proximity analysis system 120, in addition to the signal data. As explained herein, geolocation information may in some embodiments be used for geo-fencing purposes while not being used for contact tracing purposes (e.g., not being used for determination of time in proximity or closeness of physical distance to other devices). In some embodiments, system 100 is geo-fenced such that system 100 is configured to function only in certain geographic locations, or configured to not function in certain geographic locations. If system 100 is geo-fenced, then the analysis engine 122 may determine if location data (e.g., GPS data) indicating a location of mobile device 112 is within an approved location for performing signal data upload/logging and contact tracing (e.g., on company grounds). Analysis engine 122 may determine whether the location associated with mobile device 112 indicates that mobile device 112 was in an approved location for contact tracing at the time at which one or more signals were detected. Alternately or additionally, analysis engine may determine whether the location associated with mobile device 112 indicates that mobile device 112 was not in a forbidden location for contact tracing at the time at which one or more signals were detected. In accordance with a determination that contact tracing is not permissible based on the location data, analysis engine 122 may discard some or all of the information that proximity analysis system 120 received from mobile device 112. In accordance with a determination that contact tracing is not permissible based on the location data, analysis engine 122 may store some or all of the information that proximity analysis system 120 received from mobile device 112 in signal database 124. In some embodiments, location data may be discarded and not persistently stored regardless of whether geo-fencing criteria are satisfied, and signal data (e.g., signal data) may be persistently stored only when geo-fencing criteria are satisfied.

Analysis engine 122 is connected to the signal database 124. The analysis engine 122 may perform contact tracing for one or more users. In some embodiments, the analysis engine 122 is triggered to perform contact tracing when the system receives a request to trace contacts of a specific user. In some embodiments, a request to trace contacts of a specific user may be triggered by the system receiving an indication that the user has tested positive for a disease, that the user has been exposed to someone who has tested positive for a disease, or that the user otherwise has a particularized risk of disease. The system may be configured to accept self-reporting for positive disease testing or potential exposure from users and/or to allow system administrators to execute an input indicating a positive test or potential exposure for a user.

In some embodiments, analysis engine 122 retrieves signal data from signal database 124. Analysis engine 122 applies one or more algorithms to the retrieved signal data in order to determine which users have been in contact with or proximate to an infected or potentially infected user. The analysis engine 122 may quantify and/or characterize a risk (e.g., an exposure risk, disease risk), for example by generating a proximity score, for other users and may optionally generate one or more notifications based on the calculated risk. In some embodiments, quantifying and/or characterizing a risk (e.g., an exposure risk and/or a disease risk) may include generating a “proximity score” quantifying/characterizing time in proximity and/or closeness of physical proximity (e.g., distance).

In some embodiments, calculation of a proximity score (and/or of a risk level) may be based on the signal data indicating an overlapping set of signals detected by two devices. For example, if two devices each detect an overlapping set of signals with sufficient signal strength within a predetermined time window, then the system may determine that a risk exists or that a risk event has occurred. In some embodiments, the system may determine whether a predefined percentage of overlapped signals, calculated for example using a Jaccard Index, exceeds a predefined threshold; if the predefined threshold is exceeded, then the system may determine that a risk exists or that a risk event has occurred.

In some embodiments, the system may determine whether the signal strength, duration of overlap, temporal spacing of overlap, and/or percentage of overlapping signals exceeds one or more predefined thresholds, and the system may classify the exposure event into predefined categories based on the predefined thresholds. For example, each risk event may be categorized as high risk, medium risk, or low risk.

In some embodiments, the system may be configured such that, when it is determined that a risk event has occurred (e.g., when overlapping signals satisfy predefined criteria), then a count may be iterated. In some embodiments, a count may be compiled in a score vector that sums the number of high risk events, medium risk events, and low risk events in each of three respective vector components ([h, m, l]).

In some embodiments, the system may then calculate an overall risk classification or proximity classification based on one or more weighted sums of the score vector components. The high-risk vector component may be weighted most heavily while the low-risk vector component may be weighted least heavily. The weighted sum of the vector components may be compared to one or more predefined thresholds to determine whether the system should indicate an overall “high risk”/“high proximity score,” “medium risk”/“medium proximity score,” or “low risk”/“low proximity score.”

In one specific example, a risk event may be determined to have taken place if two devices detect an overlapping set of WiFi signals with RSSI greater than or equal to −75 more than 15 minutes apart. The risk event may be calculated as a high-risk event if the Jaccard Index of overlapping signals exceeds 0.6, a medium-risk event if the Jaccard Index of overlapping signals exceeds 0.5, and a low-risk event if the Jaccard Index of overlapping signals exceeds 0.4. A score vector [h, m, l] may be iterated by counting the number of risk events falling into each of the three buckets. An overall proximity score may then be determined by determining a “high risk”/“high proximity score” if there are at least 15 counts in the h vector component; a “medium risk”/“medium proximity score” if the weighted sum (1.5*h)+(m) is greater than or equal to 15; and a “low risk”/“low proximity score” if the weighted sum (3*h)+(1.5*m)+(l) is greater than or equal to 15.

In another specific example, a risk event may be determined to have taken place if two devices detect the same BLE with RSSI greater than or equal to −55 more than one minute apart. The risk event may be calculated as a high-risk event if the Jaccard Index of overlapping signals exceeds 0.65, a medium-risk event if the Jaccard Index of overlapping signals exceeds 0.45, and a low-risk event if the Jaccard Index of overlapping signals exceeds 0.3. A score vector [h, m, l] may be iterated by counting the number of risk events falling into each of the three buckets. An overall proximity score may then be determined by determining a “high risk”/“high proximity score” if there are at least 15 counts in the h vector component; a “medium risk”/“medium proximity score” if the weighted sum (1.5* h)+(m) is greater than or equal to 15; and a “low risk”/“low proximity score” if the weighted sum (3*h)+(1.5*m)+(l) is greater than or equal to 15.

In some embodiments, interpolation may be used to fill in data gaps. For example, for the above example which applies a one-minute time criteria for overlapping BLE signals, interpolation may be used to fill in data gaps of more than one minute and less than 4 minutes, with the average RSSI before and after the data gap. For example, if a device detected another device at 11:45 AM with −75 RSSI, and then detected the same device at 11:49 M with −77 RSSI, interpolation could be used to create observations for 11:46 AM, 11:47 AM and 11:48 AM with −76 RSSI.

In some embodiments, a single system such as proximity analysis system 120 may be configured for simultaneous use in more than one system for automatic contact tracing. Thus, while proximity analysis system 120 may be a part of system 100 as shown, the same proximity analysis system 120 may also be a part of other automatic contact tracing systems for tracking potential vectors of a disease outbreak in other environment, and proximity analysis system 120 may function in a same or similar manner (as described in further detail herein) for those other systems as it does for system 100. For example, system 120 (or a component thereof such as engine 122) may provide risk determination, proximity score calculation, and/or contact tracing functionality to multiple distinct enterprise-level deployments at the same time. Data collected as part of different enterprise deployments may be segregated by system 120 in order to preserve participant privacy and security.

System 100 further comprises web application components 130. Web application components 130 may comprise one or more processors configured to provide web application 134 that may be accessed by uses (e.g., enterprise level users) via dashboard 132, which may include one or more graphical user interfaces for interacting with system 100 for performing contact tracing operations. As described further below, dashboard 132 may provide a graphical user interface (GUI) that may be used by system administrators or authorized users to input, view, and access information about infected and/or potentially exposed employees and any results of contact tracing performed for said employees. Dashboard 132 may be displayed on devices including: a computer, a tablet, a mobile phone, or any device configured for graphical displays.

Web application components 130 may further comprises one or more computer storage devices (e.g., databases) used for an employee information database 136 and an account management system 138. In some embodiments, web application 134 may be configured to communicate with employee information database 136 ad/or account management system 138 in order to query, look up, store, access, or cross-correlate employee information as part of a contact tracing inquiry. For example, as described herein, employee information may be looked up in employee information database when signal data stored in database 124 needs to be correlated with an employee in order to perform contact tracing operations and/or generate alerts/notifications regarding potentially exposed individuals.

Employee information database 136 is configured to store information regarding the users in system 100. User information included in the employee information database 136 may comprise: first name, last name, email address, phone number, personal contact information, birthday, employee identifiers, device identifiers, and/or user login username. The one or more computer storage devices used by the employee information database 136 may also include user information modification and/or retrieval processes disclosed herein and/or data (e.g., configurations and/or settings for systems and/or devices, historical logs, etc.) regarding a one or more employee information database 136.

The account management system 138 is configured to perform user authentication. The account management system 138 may comprise one or more computer storage devices (e.g., databases) used to store user account information. User account information may include: username, password, and/or access rights (e.g., whether a user is able to view and/or modify the dashboard 132). The one or more computer storage devices associated with the account management system 138 may also include instructions for user authentication and/or data (e.g., configurations and/or settings for systems and/or devices, historical logs, etc.) regarding the account management system 120. (Additionally or alternatively, said information may be stored in database 136.)

Account management system 138 is connected to the employee information database 136. In some embodiments, when a user registers for the system 100, the account management system 138 verifies the eligibility of the user. The account management system 138 may verify the eligibility of a user, for example, by verifying the user is registering using an eligible email address (e.g., a corporate or institute email address), and/or by verifying that the provided user information meets predetermined requirements (e.g., age requirements, geolocation requirements, or any other requirements that may be verified by the user registration information). If the user account is eligible, the account management system 138 may send the user account information to the employee information database 136, and the employee information database 136 may add a corresponding entry for the user.

Account management system 138 may also authenticate an existing user's log-in information and access rights. The account management system 138 may authenticate a user's log-in attempt by verifying the user's username and password information. The account management system may also be configured to retrieve a user's access rights, and to determine whether the user is able to view and/or interact with dashboard 132.

Web application 134 may connect to employee information database 136 and account management system 138. In some embodiments, web application 134 is configured to facilitate the authentication process for authorized users to access and interact with system 100 via dashboard 132. When a user attempts to log in to access dashboard 132, the user's credentials (e.g., username and password) may be provided to the account management system 138, the account management system 138 and the authentication result may be returned for the web application to determine whether to allow the user to access dashboard 132.

In some embodiments, web application 134 may act as a broker between employee information database 136 and proximity analysis system 120. Web application 134 may be cloud-hosted and may process information inputted as part of a dashboard query by a dashboard user. For example, web application 134 may map an email address of a target user (inputted by the dashboard user) to the AdID and/or UUID of the target user's mobile electronic device and may pass the AdID and/or UUID (along with a date/time range inputted by the dashboard user as part of a query) to proximity analysis system 120 for performing contact tracing. When contact tracing results are returned by proximity analysis system 120, web application 134 may then use employee information database 136 to map the AdIDs and/or UUIDs of the mobile electronic devices indicated as having been in contact with the target user's device to user information (e.g., names, email addresses, and/or offices) for users of those other devices. Web application 134 may then transmit the contact tracing analysis results to dashboard 132 for visualization.

In some embodiments, the contact analysis results data is not stored in the web application 134, nor in any database of the system, but is instead automatically discarded as soon as the user of dashboard 132 closes the window of the analysis results or otherwise terminates their session.

To perform contact analysis on a target user (e.g., an infected or exposed employee), web application 134 may retrieve information from employee information database 136. In some embodiments, web application 134 may retrieve a target user's device identifiers associated with a user email address from employee information database 136. Web application 134 may send the target user's information, along with the contact tracing parameters to proximity analysis system 120. When the analysis result (e.g., a list of AdIDs and/or UUIDs of the mobile electronic devices that have been proximate to the target user's mobile device) comes back from the proximity analysis system 120, the web application 134 maps the AdIDs and/or UUIDs of the mobile electronic devices that have been proximate to the target user's mobile device to employee names and offices based on the data stored in employee information database 136.

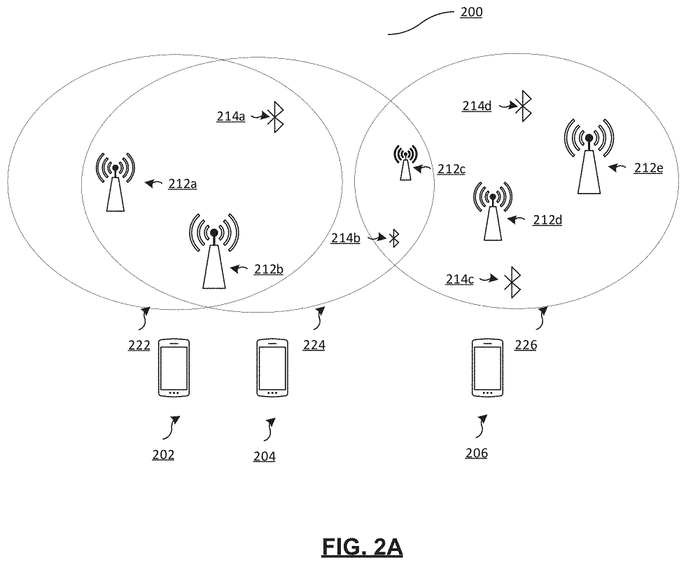

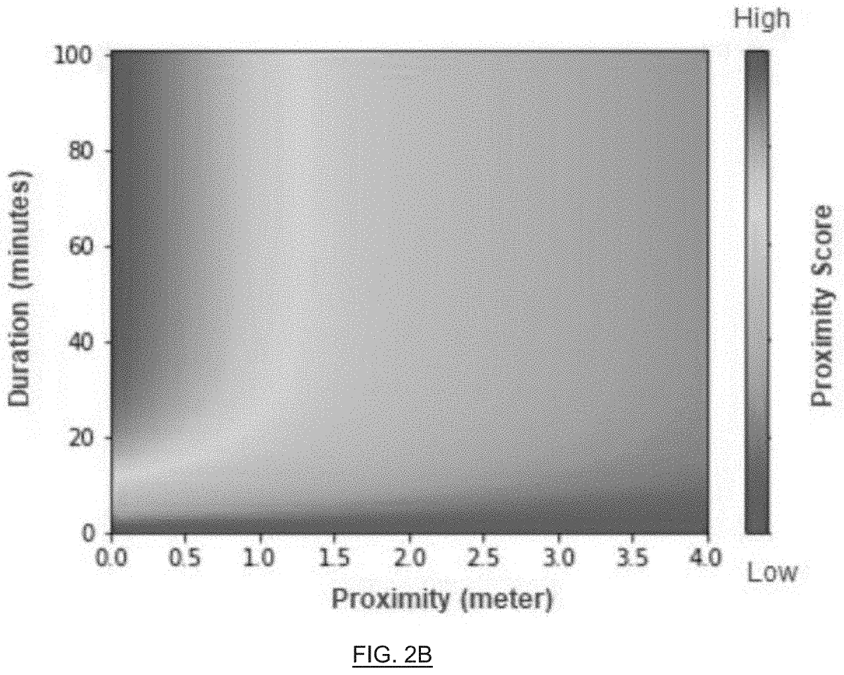

FIG. 2A shows an example of multiple mobile electronic devices deployed in system 200, in accordance with some embodiments. In some embodiments, devices 202, 204, and/or 206 may share any one or more characteristics in common with devices 110 described above with respect to system 100. In some embodiments, system 200 may generate a quantification/characterization of risk (e.g., exposure risk or disease risk), for example a determination of a proximity score, for example as described above with respect to system 100.

In the example of FIG. 2A, the system 200 comprises three mobile electronic devices. Device 202 detects electromagnetic signals in the range 222, including signals from Wi-Fi access points 212a-b, and Bluetooth device 214a. Device B 204 detects electromagnetic signals in the range 224, including signals from Wi-Fi access points 212a-c, and Bluetooth devices 214a-b. Device 206 detects electromagnetic signals in the range 226, including signals from Wi-Fi access points 212c-e, and Bluetooth devices 214b-d.

In the example of FIG. 2A, the system 200 may determine that the users of device 202 and device 204 have been proximate to one another (e.g., a “high proximity” level or a “medium proximity” level), as device 202 and device 204 have both detected signals from Wi-Fi access points 212a-b and Bluetooth device 214a at the same time. Meanwhile, system 200 may determine that device 204 and device 206 have been proximate to one another, but to a lesser extent than the proximity/contact between device 202 and device 204; this determination may be based on device 204 and device 206 detecting a smaller number of overlapping signals (e.g., signals from 212c and 214b but not from other sources). For example, system 200 may determine that device 204 and device 206 have a “medium proximity” level or a “low proximity” level. Finally, system 200 may determine that the users of device 202 and device 206 have not been in contact, as device 202 and device 206 have not detected overlapping electromagnetic signals at the same time (and/or within a predetermined time period of one another.

In some embodiments, device 202 and device 204 may detect Bluetooth signals transmitted by each other with high signal strength, while device 206 may not be able to detect the Bluetooth signals transmitted by device 202 and device 204 or the signal strength is low. This inter-device signal strength may be used in determining an extent of contact/proximity (e.g., determining a proximity score) in addition to or alternatively to signal identity, duration of detection, and signal strength detected from other devices.

FIG. 2B shows an example of a heat map of a proximity score calculated based on duration of physical proximity and on closeness (e.g., physical distance) of physical proximity. As shown, a longer duration (continuous and/or in multiple bursts) may lead to a higher proximity score (which may correspond to a higher disease risk or a higher exposure risk) being calculated. As shown, closer physical proximity distance may lead to a higher proximity score (which may correspond to a higher disease risk or a higher exposure risk) being calculated. In some embodiments, a proximity score may be calculated as a numerical quantification (e.g., between 0 and 1). In some embodiments, proximity scores may be sorted into classifications (e.g., buckets) such as “high proximity score,” “medium proximity score,” “low proximity score,” and “no proximity score.” In one example, the following schema may be used:
High proximity score: Exposed user was in close contact (e.g., within predetermined distance) with the infected user for at least a predetermined amount of time (e.g., 30 minutes total duration).
Medium proximity score: Exposed user was in some degree of physical proximity (e.g., within a predetermined distance) with the infected user but either the distance between them was greater or the contact time was shorter than the criteria for a high proximity score.
Low proximity score: Exposed user was in some degree of physical proximity with the infected user (e.g., within a predetermined distance) but either the distance between them was greater or the contact time was shorter than the criteria for a medium proximity score. Alternatively, the exposed user was in the same location as the infected user within a predetermined window of time, though not at the same time.
   In some embodiments, a system may be configured to determine whether to take one or more automated actions (e.g., whether to generate an alert) based on whether a user's proximity score is categorized into a predefined category (e.g., “high proximity score”).

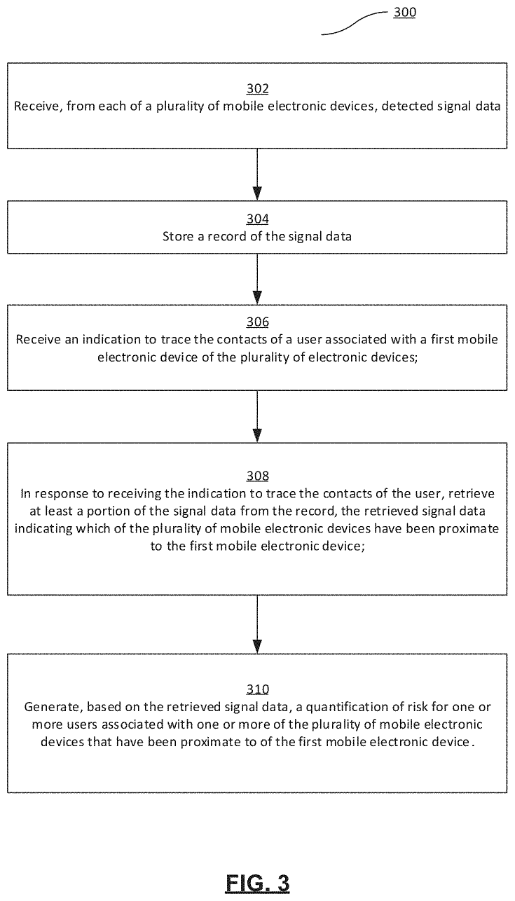

FIG. 3 shows a flowchart showing a method 300 for performing automatic contact tracing, in accordance with some embodiments. In some embodiments, method 300 may be performed by a system such as system 100 as discussed above with respect to FIG. 1 or system 200 as discussed above with respect to FIG. 2A.

At block 302, in some embodiments, the system receives, from each of a plurality of mobile electronic devices, detected signal data. In the example of system 100, the mobile electronic devices 110 may detect a plurality of electromagnetic signals. As discussed above, said signals may be emitted by other users' mobile electronic devices. Said electromagnetic signals, such as WiFi signals and/or Bluetooth signals, may only be able to be detected when the emitting device and the listening device are within a certain physical proximity (e.g., certain physical distance) of one another. The detection of said electromagnetic devices may therefore be taken as an indication that the devices are within a certain estimated physical proximity (e.g., certain physical distance) of one another at the time of the detection. In some embodiments, detection of a signal from another mobile electronic device may be noted; in some embodiments, a strength of a signal detected from another mobile electronic device may be noted, such that the signal strength may thereafter be used to inform a calculation of estimated physical proximity (e.g., estimated physical distance) between the two devices.

As described elsewhere herein, signals may be collected from other mobile electronic devices (e.g., amongst participating devices) and/or from electronic devices that are not mobile electronic devices of another user. For example, a mobile electronic device may detect signals, such as WiFi signals and/or Bluetooth signals, emitted by a WiFi hot-spot or IOT device. In some embodiments, information about signals detected from these devices may be stored (e.g., in a user's signal map and/or in a database of signal data detected by devices in the system) and used to determine a user's risk levels and/or proximity score with respect to another user. For example, if two users' mobile devices are each connected to the same WiFi hot-spot at the same time, then the system may determine that the users are in a certain physical proximity to one another (e.g., within a distance of at most twice the estimated signal range of the WiFi hot-spot), even if neither of those users' mobile devices detect any signals emitted by one another directly. In some embodiments, the system may be configured to treat one or more fixed electronic devices aside from a mobile electronic device as a hub for the purposes of contact tracing. For example, in addition to enabling mobile electronic devices to broadcast identity information and to listen for signals emitted by other devices, the system may alternatively or additionally enable one or more fixed electronic devices to broadcast identity information and/or to listen for signals emitted by other devices. Fixed electronic devices may include, for example, desktop computers, WiFi hot spots, routers, AV equipment, IOT devices, or the like; in some embodiments, a fixed electronic device may be identified based on a Wireless AP MAC ID, rather than a device UUID or AdID. In this manner, fixed electronic devices may collect signal data that may be used for contact tracing, wherein a fixed physical space (e.g., a conference room in which a certain AV device is located) may be treated as an entity for the purpose of contact tracing. In this way, contacts between pairs of human users associated with respective mobile electronic devices may be traced, and contacts between a human user and a physical space or fixed electronic device may also be similarly traced using the methods described herein.

At block 304, in some embodiments, the system stores a record of the signal data. In the example of system 100, the signal data and related metadata may be stored in signal database 124. In some embodiments, data collected during collection of signal data signals may include one or more of:
Mobile device information (e.g., phone manufacturer and model; mobile OS identity and/or version; contact tracing application name and/or version; GPS coordinates (for geofencing purposes only); and/or Mobile App-generated unique identifier assigned to user's device to identify device for contact tracing);
Signal data (e.g., observed WiFi/Bluetooth MAC addresses, SSIDs, BLE UUIDs, Major and Minor (BLE identifiers), BLE RSSI (Receiver Signal Strength Indicators), and/or Longitude/Latitude information, including time of detection and/or other metadata for any of said data);
Analytics data (e.g., performance monitoring and cookie data); and
Diagnostic data (e.g., periodic battery and cellular connectivity status updates to help diagnose device performance issues).

In some embodiments, the signal data may be uploaded for storage in a database by WiFi connection or by other internet connection. In some embodiments, the signal data may be stored along with associated metadata. The metadata may include, for example, time of detection, time of storage, device type used to collect the data, etc.

In some embodiments, signal data may be stored in association with a user identifier such as an identifier used to identify users, devices, or other assets (e.g., a device UUID). (In some embodiments, an identifier may be an identifier used for the purposes of advertisement services, such as an AdID.) In some embodiments, signal data may be stored separately from personal information about users (e.g., user name, user address, user demographic information, etc.), such as by maintaining personal information in a separate database (e.g., database 136). In some embodiments, signal data may be maintained by a third-party service and/or segregated infrastructure that does not have access to user personal information, in order to increase user privacy. As used herein, upload and storage of signal data may refer to upload and storage of data regarding detected signals (e.g., signal identity, signal content, signal type, signal strength, time of detection). This data regarding detected signals may thereafter be used to calculate a proximity score indicating information about whether (and the extent to which) one or more devices (or associated persons) are estimated to have been proximate to one another.

In some embodiments, signals that are detected may be used to construct a “signal map” for a device, and the signal map may be used to calculate an estimation of the device's physical proximity (e.g., physical distance) to one or more other electronic devices based on the strength and identity of the signals detected. The signal map may comprise information about the identity, strength, configuration, and timing of signals detected by a mobile electronic device. In some embodiments of the example of system 100, a signal map may be stored in database 124.

As discussed above, in some embodiments, the system may be configured to be geo-fenced. In some embodiments in which the system is geo-fenced, the system is configured such that mobile electronic devices gather and upload signal data regardless of location, but that the signal data is uploaded along with location data indicating a location of the uploading mobile electronic device, such that the signal data may be stored only when the location data indicates that the uploading mobile electronic device is in an approved area such as on company grounds. In some embodiments in which location data is uploaded for the purposes of verifying that geo-fencing criteria is met, the uploaded location data may be deleted and may not be stored persistently by the system (regardless of whether the location data indicates at geo-fencing criteria are met).

In some embodiments, in addition to or alternatively to enforcing geo-fencing criteria at the data storage stage as described above, the system may enforce geo-fencing criteria at the data broadcast stage and/or the data collection stage, such that signals for use in risk determinations and/or proximity score determinations (e.g., BLE and/or RSSI) may only be broadcast when it is first determined based on location data that geo-fencing criteria are satisfied, or such that signal data may only be detected and/or recorded at the mobile electronic device when it is first determined based on location data that geo-fencing criteria are satisfied.

In some embodiments, one or more additional criteria, in addition to geo-fencing criteria, may be applied before a device broadcasts information, a device detects or uploads information, or the system stores information. For example, in some embodiments, a mobile electronic device (e.g., smartphone) may only broadcast and/or detect information for contact tracing when an application associated with the system is actively running on the device. In some embodiments, a mobile electronic device may only collect information if the device is determined to be in a location having sufficient LTE-M coverage to allow for signal data backhaul at a predefined minimum frequency (e.g., at least once per day).

At block 306, in some embodiments, the system receives an indication (e.g., an instruction or request) to trace the contacts of a user associated with a first mobile electronic device of the plurality of electronic devices. In some embodiments, a request or instruction may be received by the system via an explicit user request, a pre-scheduled operation, or a programmatically-triggered operation (e.g., one or more predetermined or dynamically-determined trigger conditions being met). In some embodiments, the system may be configured to not perform any programmatically/automatically triggered contact tracing, such that an explicit user-generated instruction to perform contact tracing operations may be required. In some embodiments, users may be able to use an API to execute an instruction to perform contact tracing operations.

In some embodiments, performing contact tracing for one or more users may be triggered by the system receiving a request to trace contacts of a specific user. A request to trace contacts of a specific user may be triggered by the system receiving an indication that the user has tested positive for a disease, that the user has been exposed to someone who has tested positive for a disease, or that the user otherwise has a particularized risk of disease. In some embodiments, the system may be configured to accept self-reporting for positive disease testing or potential exposure from users and/or to allow system administrators to execute an input indicating a positive test or potential exposure for a user. In the example of system 100, the indication to trace the contacts of a user may be input executed via dashboard 132.

At block 308, in some embodiments, in response to receiving the indication to trace the contacts of the user, the system retrieves at least a portion of the signal data from the signal database 124, the retrieved signal data indicating which of the plurality of mobile electronic devices have been proximate to the first mobile electronic device (e.g., the data indicating which devices meet signal identity, signal strength, signal detection time, and/or signal detection duration criteria). In some embodiments, retrieving the signal data for a user may require the system to first obtain the user identifier (e.g., AdID) used by the database storing the signal data, such that the system may identify the relevant signal data to extract from the database. In the example of system 100, database 124 stores the signal data. In some embodiments, the system may look up the identifier associated with the user in a separate database (e.g., database 136 in system 100), such as an enterprise database maintained separately from the database storing the signal data.

In some embodiments, the system may trace the contacts of a user by retrieving signal data associated with the target user over a time period associated with the target user's infection or potential exposure. For example, a request to trace user contacts may specify a period of time over which contacts should be traced, and signal data associated with that time window may then be retrieved. In some embodiments, the system may determine a window over which to retrieve signal data based on a disease diagnosis or potential disease exposure for the user; for example, the time period may be determined based on an incubation period or potential incubation period of a disease for which a user has tested positive. In some embodiments, the system may allow the dashboard user instructing the search to input parameters for the contact tracing process. The contact tracing parameters may include the earliest date to retrieve signal data for, the latest date to retrieve signal data for, a period length to retrieve signal data for, and/or the type of signal to be used for risk determination and/or proximity score calculation.

At block 310, in some embodiments, the system generates, based on the retrieved signal data, a quantification and/or characterization of risk (e.g., disease risk or exposure risk) for one or more users associated with one or more of the plurality of mobile electronic devices that have been within the predefined proximity of the first mobile electronic device. The quantification of risk may include a numerical score (e.g., a risk score or a proximity score and/or a classification (e.g., “high risk,” “medium risk,” “low risk;” or “high proximity, “medium proximity,” or “low proximity”).

In some embodiments, a quantification/characterization of a risk (e.g., exposure risk or disease risk) may include and/or be provided as a quantification or characterization of proximity, such as a proximity score. The quantification/characterization may be calculated in accordance with one or more predefined algorithms and/or using one or more machine learning algorithms. In some embodiments, the calculation may be based on a number of times a user was proximate to an exposed user, a time at which a user was proximate to an exposed user, a length of time over which a user was proximate to an exposed user, and/or an closeness of physical proximity (e.g., physical distance, for example as calculated based on signal strength) between a user and an exposed user and/or between devices with which the user and the exposed user are associated. In some embodiments, a risk level may be calculated with respect to a specific disease and/or pathogen, and the system may be configured to calculate different risk levels for a single user for different diseases and/or different pathogens.

In some embodiments, the system may be configured to apply one or more algorithms to quantify/characterize risk and/or an extent of contact/proximity, for example to determine which users should be classified as having a high proximity score, medium proximity score, or low proximity score with respect to the target user. In some embodiments, the calculated proximity score may be a function of one or both of (i) the duration of signal overlap and/or cross-device signal connection and (ii) an estimated physical proximity (e.g., physical distance) for example as estimated or calculated based on overlapping/cross-detected signal strength and/or number of overlapping signals for the two users. For example, duration of contact may be estimated based on observed overlapping time between two mobile electronic devices and may be incorporated with frequency of overlap. Physical closeness of location may be estimated by signal strength (e.g., RSSI) of BLE signals transmitted by nearby mobile electronic devices. If a second user's mobile electronic device observes a similar set of ambient WiFi and/or Bluetooth signals as an infected user's mobile electronic device for an overlapped time, or the second user's mobile electronic device detects the infected user's BLE signal, the system may determine that the second user has been in contact with the infected user. In some embodiments, a second user may be determined to be at a higher risk (e.g., higher proximity score) if the second user's mobile electronic device detected similar and/or overlapping signals with an infected user with a higher signal strength and/or for a longer period of time.

In one example, proximity characterizations and/or risk characterizations may classify one or more users/devices into “high proximity score,” “medium proximity score,” and “low proximity score” categories as follows. A high proximity score may be assigned when a second user's mobile electronic device scanned similar ambient WiFi/Bluetooth signals as those signals scanned by a target user's mobile electronic device, and/or the second user's mobile electronic device received a strong signal (e.g., above a predetermined or dynamically determined signal strength threshold) via BLE transmission from a target user's mobile electronic device for more than a predetermined or dynamically determined threshold amount of time (e.g., 30 minutes), with the threshold amount of time calculated either continuously or intermittently, during a date/time range specified via the system dashboard. A medium proximity score may be assigned when a second user's mobile electronic device detects signals that overlap with signals detected by a target user (e.g., overlapping in identity, strength profile, and/or time) in a significant manner in a first instance but in an insignificant manner in a second instance. A medium proximity score may also be assigned when a signal strength of a BLE signal detected from a target user's mobile electronic device is strong in one instance but is weak in another instance. A low proximity score may be assigned when ambient WiFi/Bluetooth/BLE signals detected by a second user's mobile electronic device have any non-zero signal overlap with the signal scans detected by (or BLE signals broadcast from) the target user's mobile electronic device within a predefined or dynamically determined time window (e.g., 4 hours). In some embodiments, users not meeting the criteria for high, medium, or low proximity score may not be assigned any proximity score. In some embodiments, for example as explained above, a determination of an overall proximity score or risk level may be based on a weighted sum of vector components of a vector, wherein the vector comprises components that are based on (e.g., iterated as counts based on) signal-overlap events that meet signal-data criteria to be classified as high-risk events, medium-risk events, or low-risk events.

Method 300, as described above, contemplates that signal data may be collected by user's mobile electronic devices based, in part, on direct detection of other user's mobile electronic devices (e.g., smartphones), such that a single device (e.g., smartphone) may serve to both broadcast and collect data. In some embodiments, additionally or alternatively, users in contact tracing systems such as those described herein may carry personal beacon devices that may be separate from a detection device (e.g., separate from a smartphone device). In some embodiments, a personal beacon device may be a dedicated broadcast device that serves to broadcast signals but not to detect, collect, or record signals. In some embodiments, a personal beacon device may be associated with a device ID (e.g., UUID) and may be associated with a beacon session in the system, linking the personal beacon device to a user's identity and to a personal beacon BLE UUID. As a person carrying the personal beacon device navigates the contact tracing environment (e.g., the organization's geo-fenced sites), existing mobile electronic device applications and/or contact tracing devices may observe the personal beacon by detecting the signals that are broadcast from the personal beacon, said signals including BLE UUID and/or and RSSI. Those detected signals may be uploaded by the detecting devices (optionally subject to geo-fencing criterial) for storage in a signal database and for use in risk/proximity characterizations/quantifications as described herein.

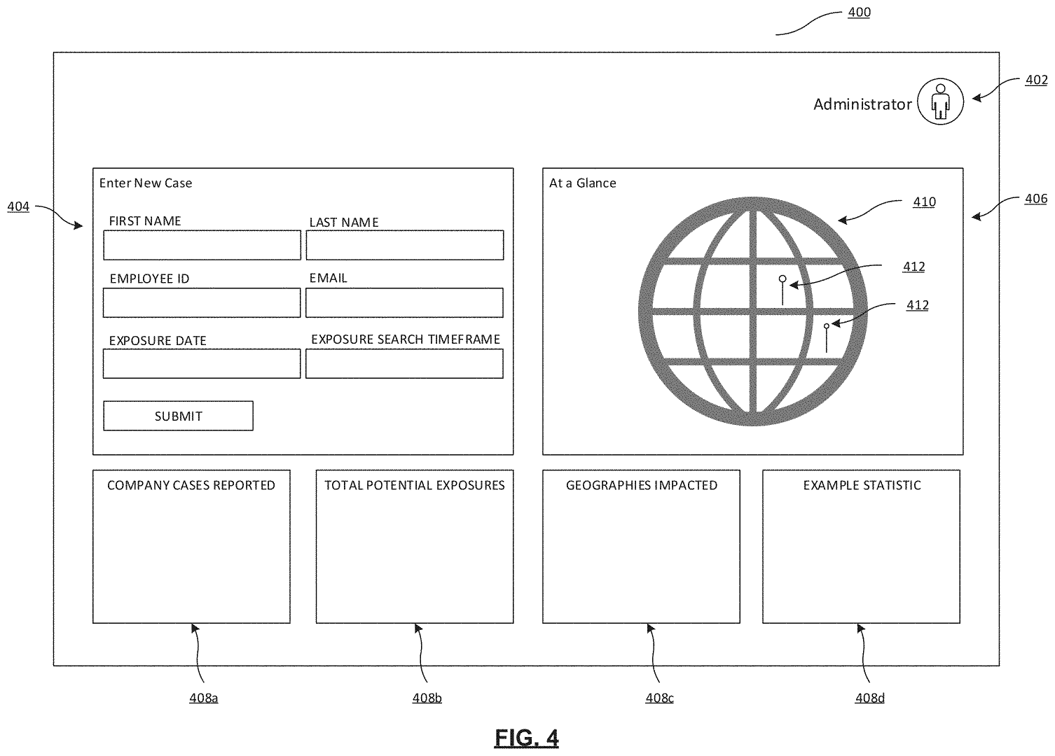

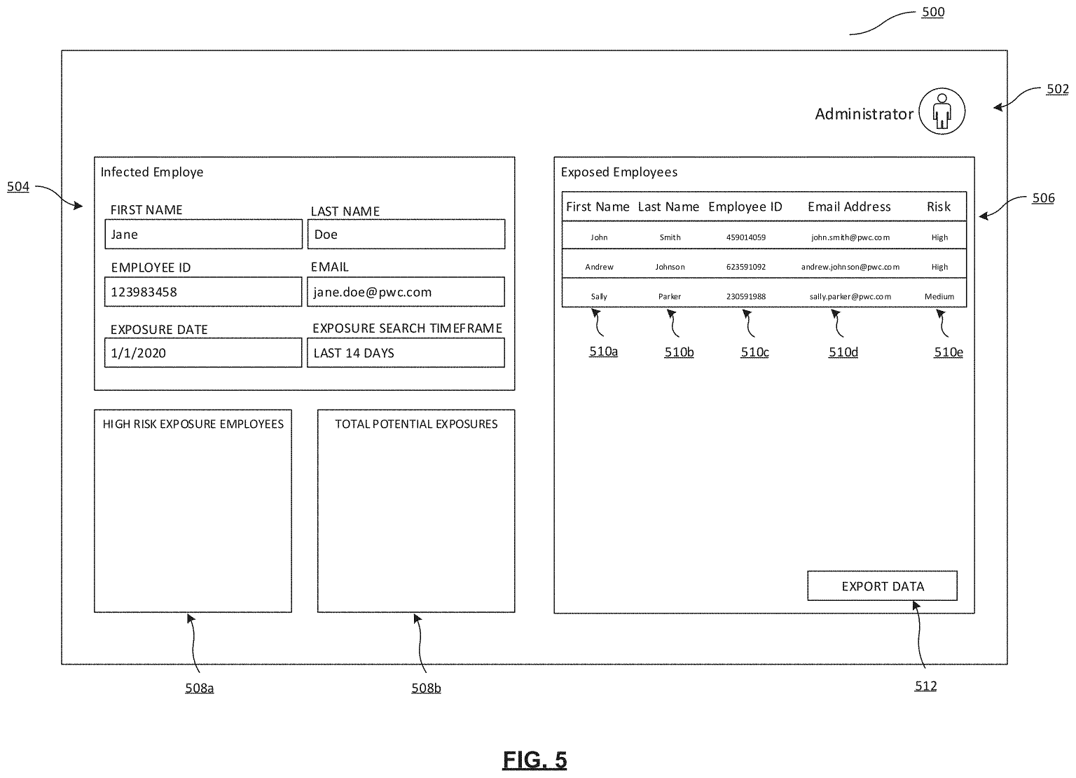

FIGS. 4 & 5 show screens of a graphical user interface (GUI) for an automatic contract tracing system, in accordance with some embodiments. In some embodiments, the GUI screens shown in FIGS. 4 and/or 5 may be caused to be displayed by system 100 and/or system 200. In the example of system 100, the GUI screens shown in FIGS. 4 and/or 5 may be displayed as part of dashboard 132. In the embodiments shown in FIGS. 4-5, the screens of the GUI are configured for display and use via a desktop or laptop device, including by clicking on various selectable icons in order to access GUI functionality. However, same or similar screens and/or functionalities may be displayed and/or used by touch-screen devices, such as tablet devices and/or smart phone devices, and the various selectable icons shown may in some embodiments be selected by tapping on the icons on a touch-screen device.

FIG. 4 shows screen 400, which is a home screen, in accordance with some embodiments. In some embodiments, a home screens such as screen 400 may be a landing page for a GUI for managing an enterprise-level deployment of an automatic contact tracing system, such as those described herein.

In some embodiments, the system may provide screen 400 for enterprise-level users (e.g. human-resources administrators, in the example of an enterprise-deployed system) to input, view, and access information about infected and/or potentially exposed employees and any results of contact tracing performed for said employees. Enterprise-level dashboard users may be able to configure one or more enterprise-wide and/or user-specific data collection preferences, for example in order to comply with different data privacy regulations in different regions. Enterprise-level dashboard users may be able to configure one or more enterprise-wide and/or user-specific contact-tracing sensitivity parameters, such as a level of risk that will trigger an alert and/or a preference for whether (and to what extent) to engage in higher-order contact tracing for users with direct or indirect exposures.

As shown, screen 400 comprises a selectable admin icon 402. The selectable admin icon 402 may be selected (e.g., clicked or tapped) to navigate to other screens of the GUI. In some embodiments, the admin icon 402 may show notification data.

As shown, screen 400 further comprises a user input area 404. User input area 404 may be used to input information of a user who has tested positive for a disease, a user who has been exposed to someone who has tested positive for a disease, and/or a user that otherwise has a particularized risk of disease.

User input area 404 may comprise areas that allow input of user identifying information. The user identifying information may include first name, last name, employee ID, email, and/or the user's device identifier. User input area 404 may further comprise areas that allow input of contact tracing search parameters. Search parameters may include exposure date and exposure search timeframe. User input area 404 may further comprise a selectable “submit” icon. In some embodiments, the selection (e.g., click or tap) of the “submit” icon triggers the system with a request to trace contacts of the user specified in the user input area.

In some embodiments, the system may be configured to accept self-reporting for positive disease testing or potential exposure from users and/or to allow system administrators to execute an input indicating a positive test or potential exposure for a user.

As shown, screen 400 further comprises data display areas 406 and 408a-d, which display information regarding the managed system (e.g., system 100). Data display area 406 may display a map 410, upon which a plurality of geolocation pins 412 may be used to indicate the locations in which users have tested positive for a disease. Data display areas 408a-d may indicate statistics of the system 100. Data display area 408a may indicate the number of cases of positive test results for the disease in the company, data display area 408b may indicate the number of users that have been potentially exposed to someone who has tested positive for a disease, data display area 408c may indicate the number of geographies (e.g., predefined geographical regions and/or dynamically-determined outbreak clusters) that are impacted by the disease, and data display area 408d may display a further statistic related to system 100.

FIG. 5 shows screen 500, which is a result screen, in accordance with some embodiments. In some embodiments, a result screen such as screen 500 may be used to display contact tracing results after a request to trace contacts of a user is submitted through screen 400.

As shown, screen 500 comprises a selectable admin icon 502. The selectable admin icon 502 may be selected (e.g., clicked or tapped) to navigate to other screens of the GUI. In some embodiments, the admin icon 502 may show notification data.

As shown, screen 500 further comprises a search parameter area 504. Search parameter area 504 may display the information submitted on screen 400 that was used to trigger the system to perform the contact tracing. Search parameter area 504 may indicate the infected user's first name (“Jane”), last name (“Doe”), employee ID (“123983458”), and email (jane.doe@pwc.com). Search parameter area 504 may further indicate the contact tracing search parameters exposure date (“1/1/2020”), and exposure search timeframe (“last 14 days,” which may be defined with respect to the exposure data and/or with respect to the query date).

As shown, screen 500 further comprises display areas 508a-b. Data display area 508a may display the number of high risk employees, and 508b may display the number of total potential exposures.

As shown, screen 500 may further comprise a contact tracing results display 506. In some embodiments, contact tracing results display 506 may be a table with columns 510a-e. Column 510a may display the first names, 510b may display the last names, 510c may display the employee IDs, 510d may display the email addresses, and 510e may display a quantification/characterization of risk and/or a proximity score. Contact tracing results display 506 may further comprise a selectable icon 512. Selectable icon 512 may be selected to export the data from contact tracing results display 506.

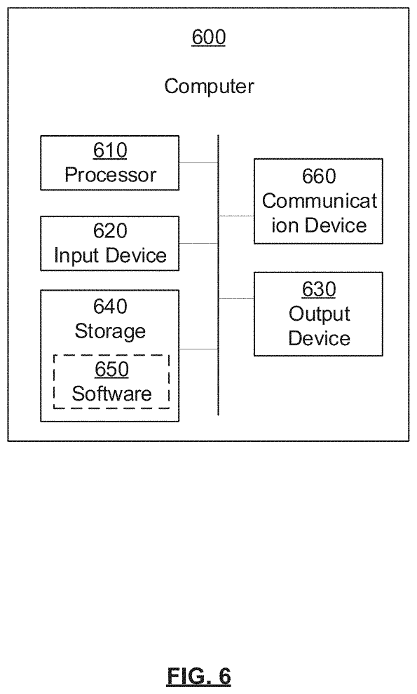

FIG. 6 illustrates an example of a computer, according to some embodiments. Computer 600 can be a component of an automatic contract tracing system according to the systems and methods described above, such as system 100 of FIG. 1. In some embodiments, computer 600 may execute a method for performing automatic contact tracing.

Computer 600 can be a host computer connected to a network. Computer 600 can be a client computer or a server. As shown in FIG. 6, computer 600 can be any suitable type of microprocessor-based device, such as a personal computer, workstation, server, or handheld computing device, such as a phone or tablet. The computer can include, for example, one or more of processor 610, input device 620, output device 630, storage 640, and communication device 660. Input device 620 and output device 630 can correspond to those described above and can either be connectable or integrated with the computer.

Input device 620 can be any suitable device that provides input, such as a touch screen or monitor, keyboard, mouse, or voice-recognition device. Output device 630 can be any suitable device that provides an output, such as a touch screen, monitor, printer, disk drive, or speaker.

Storage 640 can be any suitable device that provides storage, such as an electrical, magnetic, or optical memory, including a random access memory (RAM), cache, hard drive, CD-ROM drive, tape drive, or removable storage disk. Communication device 660 can include any suitable device capable of transmitting and receiving signals over a network, such as a network interface chip or card. The components of the computer can be connected in any suitable manner, such as via a physical bus or wirelessly. Storage 640 can be a non-transitory computer-readable storage medium comprising one or more programs, which, when executed by one or more processors, such as processor 610, cause the one or more processors to execute methods described herein.

Software 650, which can be stored in storage 640 and executed by processor 610, can include, for example, the programming that embodies the functionality of the present disclosure (e.g., as embodied in the systems, computers, servers, and/or devices as described above). In some embodiments, software 650 can include a combination of servers such as application servers and database servers.

Software 650 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include but is not limited to, an electronic, magnetic, optical, electromagnetic, or infrared wired or wireless propagation medium.

Computer 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 600 can implement any operating system suitable for operating on the network. Software 650 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

Any of the systems, methods, techniques, and/or features disclosed herein may be combined, in whole or in part, with any other systems, methods, techniques, and/or features disclosed herein.

1. A system for performing automatic contact tracing for monitoring disease outbreaks, the system comprising:
a plurality of mobile electronic devices, each of the plurality of mobile electronic devices configured to detect signal data indicating when it is proximate to another one of the plurality of mobile electronic devices;
one or more processors configured to:
receive, from each of the plurality of mobile electronic devices, detected signal data;
store a record of the signal data;
receive an indication to trace the contacts of a first user associated with a first mobile electronic device of the plurality of electronic devices;
in response to receiving the indication to trace the contacts of the first user, retrieve at least a portion of the signal data from the record, the retrieved signal data indicating which of the plurality of mobile electronic devices have been proximate to the first mobile electronic device; and
generate, based on the retrieved signal data, a quantification of risk for one or more other users associated with one or more of the plurality of mobile electronic devices that have been proximate to the first mobile electronic device.

2. The system of claim 1, wherein the indication to trace the contacts of the first user comprises an indication that the first user has tested positive for a disease.

3. The system of claim 1, wherein the indication to trace the contacts of the first user comprises an indication that the first user has potentially been exposed to a disease.

4. The system of claim 1, wherein each of the plurality of mobile electronic devices is configured to broadcast electromagnetic signals comprising identifying information able to be detected by one or more of the other mobile electronic devices.

5. The system of claim 1, wherein:
the signal data received from each of the plurality of mobile electronic devices comprises time data indicating when pairs of the mobile electronic devices were proximate to one another; and
storing the record of the signal data comprises storing the time data.

6. The system of claim 5, wherein:
the indication to trace the contacts of the first user comprises an indication of a time window for which contact tracing should be performed; and
the retrieved signal data is retrieved based on the time data stored in the record indicating that one or more of the plurality of mobile electronic devices was proximate to the first mobile electronic device during the time window indicated.

7. The system of any claim 1, comprising:
a first database in which the record of signal data is stored in association with user identifiers; and
a second database in which user personal information is stored in association with the user identifiers.

8. The system of claim 7, wherein the one or more processors are configured to, in response to receiving the indication to trace the contacts of the first user:
retrieve a first user identifier associated with the first user from the second database; and
retrieve the signal data comprises retrieving the signal data from the first database using the first user identifier.

9. The system of claim 7, wherein the user identifiers comprise user identifiers configured for use in advertisement systems.

10. The system of claim 1, wherein the one or more processors are configured to generate and transmit one or more notifications regarding the quantification of risk for one or more users.

11. The system of claim 1, wherein the quantification of risk is based on an amount of time for which the mobile electronic devices of the first user and the one or more other users were proximate to one another.

12. The system of claim 1, wherein the quantification of risk is based on a number of times that the mobile electronic devices of the first user and the one or more other users were proximate to one another.

13. The system of claim 1, wherein the quantification of risk is based on a calculated distance between the mobile electronic devices of the first user and the one or more other users that were proximate to one another.

14. The system of claim 1, wherein:
each of the plurality of mobile electronic devices is configured to detect signal data when predefined location criteria are satisfied; and
each of the plurality of mobile electronic devices is configured to not detect signal data when the predefined location criteria are not satisfied.

15. The system of claim 1, wherein the one or more processors are configured to:
receive, from each of the plurality of mobile electronic devices, location data associated with the detected signal data;
store the record of the signal data in accordance with a determination that predefined location criteria are satisfied; and
not store the record of the signal data in accordance with a determination that the predefined location criteria are not satisfied.

16. The system of claim 15, wherein the one or more processors are configured to automatically delete the received location data after determining whether the location criteria are satisfied and without regard for whether or not the location criteria are satisfied.

17. The system of claim 1, wherein the detected signal data comprises information about a detected signal broadcast from another of the plurality of mobile electronic devices.

18. The system of claim 1, wherein the detected signal data comprises information about a detected signal broadcast from a device, separate from the plurality of mobile electronic devices, including one or more of: a Wi-Fi access point, a Bluetooth device, a network-enabled appliance, a network-enabled infrastructure device, and an IoT devices.

19. The system of claim 1, wherein the record of signal data comprises, for each of the plurality of electronic devices, a signal profile generated based on a plurality of electromagnetic signals detected by a set of one or more detection antennas of the mobile electronic device.

20. The system of claim 19, wherein the plurality of electromagnetic signals comprise signals emitted one or more of: a Wi-Fi access point, a Bluetooth device, a network-enabled appliance, a network-enabled infrastructure device, an IoT device, and another of the plurality of mobile electronic devices.

21. The system of claim 1, wherein the detected signal data comprises:
a first subset of the signal data collected in accordance with a first type of signal scan performed according to a first timing schema; and
a second subset of the signal data collected in accordance with a second type of signal scan performed according to a second timing schema.

22. The system of claim 1, wherein the quantification of risk comprises one or both of: a numeric score, and a classification into a predefined risk category.

23. The system of claim 1, wherein the quantification of risk comprises one or more of: an exposure risk, a contamination risk, an infection risk, and a disease risk.

24. A method for performing automatic contact tracing for monitoring disease outbreaks, performed at a system comprising one or more processors and a plurality of mobile electronic devices, each of the plurality of mobile electronic devices configured to detect signal data indicating when it is proximate to another one of the plurality of mobile electronic devices, the method comprising:
receiving, from each of the plurality of mobile electronic devices, detected signal data;
storing a record of the signal data;
receiving an indication to trace the contacts of a first user associated with a first mobile electronic device of the plurality of electronic devices;
in response to receiving the indication to trace the contacts of the first user, retrieving at least a portion of the signal data from the record, the retrieved signal data indicating which of the plurality of mobile electronic devices have been proximate to the first mobile electronic device; and
generating, based on the retrieved signal data, a quantification of risk for one or more other users associated with one or more of the plurality of mobile electronic devices that have been proximate to the first mobile electronic device.

25. A non-transitory computer-readable storage medium comprising instructions for performing automatic contact tracing for monitoring disease outbreaks at a system at comprising one or more processors and a plurality of mobile electronic devices, each of the plurality of mobile electronic devices configured to detect signal data indicating when it is proximate to another one of the plurality of mobile electronic devices, the instructions configured to cause the system to:
receive, from each of the plurality of mobile electronic devices, detected signal data;
store a record of the signal data;
receive an indication to trace the contacts of a first user associated with a first mobile electronic device of the plurality of electronic devices;
in response to receiving the indication to trace the contacts of the first user, retrieve at least a portion of the signal data from the record, the retrieved signal data indicating which of the plurality of mobile electronic devices have been proximate to the first mobile electronic device; and
generate, based on the retrieved signal data, a quantification of risk for one or more other users associated with one or more of the plurality of mobile electronic devices that have been proximate to the first mobile electronic device.
